(19) United States
(12) Patent Application Publication
(10) Pub. No.: US 2023/0317290 A1
(43) Pub. Date: Oct. 5, 2023

(54) SYSTEMS AND METHODS FOR IDENTIFYING AND STAGING PRESSURE INJURIES

(71) Applicant: Robert Babiak, Bulverde, TX (US)

(72) Inventor: Robert Babiak, Bulverde, TX (US)

(21) Appl. No.: 18/194,429

(22) Filed: Mar. 31, 2023

Related U.S. Application Data
(60) Provisional application No. 63/326,573, filed on Apr. 1, 2022.

(51) Int. Cl. G16H 50/20; A61B 5/00; G16H 70/20

(52) U.S. Cl. CPC G16H 50/20; A61B 5/748; G16H 70/20

(57) ABSTRACT

Systems, device, and methods for staging a pressure injury are provided which may include providing a series of questions to a clinician to answer based on a provided visualization of a pressure injury. The clinician may provide answers in a yes or no format, which may be displayed with the series of questions. A final assessment may be provided based on the series of questions and corresponding answers. The questions, answers, and assessment may be provided in a visual flowchart format along with recommendations to compare the visualization of the pressure injury with a medical definition.

### CROSS-REFERENCE TO RELATED APPLICATIONS

The present application claims priority to U.S. Provisional Patent No. 63/326,573, filed on Apr. 1, 2022, the entirety of which is hereby incorporated by reference.

### TECHNICAL FIELD

The present invention relates to methods, devices, and systems for efficiently and accurately staging pressure injuries. These may include the use of staging algorithms which may allow a health care professional to answer a number of simple questions about a given pressure wound to accurately classify it and provide treatment.

### BACKGROUND

Medical clinicians, including physicians, physician assistants, nurse practitioners, nurses, physical and occupational therapists, are responsible for diagnosing and treating a variety of injuries. Pressure injuries have historically presented difficulties for clinicians to correctly stage using the National Pressure Injury Advisory Panel's (NPIAP) definitions. Mistakes in staging pressure injuries can lead to incorrect treatment of these injuries, increased costs, and delaying resolution of these injuries.

Current injury staging systems often involve multiple pages of descriptions that can require extra time to process and lead to frustration for the clinician utilizing these processes. Other systems rely on the use of the NPIAP definitions for comparing with and identifying injuries. However, this requires clinicians to have intimate knowledge of the NPIAP definitions, and without this knowledge, the clinician must review each definition to accurately identify the pressure injury. Both of these methods are time consuming and often leads to misdiagnosis of the pressure injury.

Thus, needs exist for intuitive methods for quickly assessing and staging pressure injuries.

### SUMMARY

The systems, devices, and methods disclosed herein relate generally to assessing and staging pressure injuries. A method for staging a pressure injury is provided, which may include providing a visualization of the pressure injury to a clinician; providing a series of questions to the clinician; receiving answers to each of the series of questions from the clinician; providing an assessment of the pressure injury to the clinician based on the provided series of questions and corresponding received answers from the clinician; displaying the provided series of questions and corresponding received answers along with the assessment of the pressure injury in a visual format on a single screen of a display device.

In some implementations, the visual format is a flowchart such that each of the provided series of questions is displayed in a first set of boxes and each of the corresponding received answers is displayed in a second set of boxes. The first set of boxes and the second of boxes may be connected by arrows to visually show a path of the questions and answers through the flowchart. The assessment of the pressure injury may be provided visually along with a prompt for the clinician to access a medical definition corresponding to the assessment of the pressure injury.

The assessment of the pressure injury may be provided with a medical visualization corresponding to the assessment of the pressure injury. The provided series of questions may be displayed as a set of selectable icons. The received answers to the questions may be displayed as a set of selectable icons. The provided series of questions may be in a yes or no format. In some implementations, the method may further include providing a medical recommendation based on the assessment of the pressure injury. The method may further include displaying the provided medical recommendation based on the assessment of the pressure injury with the assessment of the pressure injury in a visual format on the single screen of the display device.

A system for staging a pressure injury is also provided, which may include an electronic computer system configured to receive medical data and user input information, wherein the electronic computer system is configured to receive a visualization of the pressure injury, wherein the electronic computer system is configured to generate a series of questions related to assessing the visualization of the pressure injury; an input device for a user to input the information connected to the electronic computer system, wherein the input device is configured to receive answers to series of questions related to the assessing of the visualization of the pressure injury; and a display device configured to display the medical data and user input information in a visual format; wherein the display device is configured to display the series of questions and the received answers in a visual format on a single screen of the display device.

In some implementations, the visual format is a flowchart such that each of the provided series of questions is displayed in a first set of boxes and each of the corresponding received answers is displayed in a second set of boxes. The first set of boxes and the second of boxes may be connected by arrows to visually show a path of the questions and answers through the flowchart. The assessment of the pressure injury may be provided visually along with a prompt for the clinician to access a medical definition corresponding to the assessment of the pressure injury. The assessment of the pressure injury may be provided with a medical visualization corresponding to the assessment of the pressure injury. The provided series of questions may be displayed as a set of selectable icons. The received answers to the questions may be displayed as a set of selectable icons. The provided series of questions may be provided in a yes or no format.

A method for identifying a pressure is also provided which may include providing a visualization of the pressure injury to a clinician; providing a first question in a first icon to the clinician; receiving a first answer to the first question from the clinician; displaying the first answer to the first question in a second icon; providing a second question in a third icon to the clinician based on the first answer to the first question; receiving a second answer to the second question from the clinician; displaying the second answer to the second question in a fourth icon; providing an assessment of the pressure injury based on the provided first and second questions and received first and second answers; and displaying the assessment of the pressure injury in a visual format along with the first, second, third, and fourth icons on a single screen of a display device.

In some implementations, the method may include displaying the assessment of the pressure injury in the visual format with the first, second, third, and fourth icons in a flowchart such that adjacent icons are connected visually to show an order of questions and answers.

It is to be understood that both the foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the accompanying drawings and the following detailed description.

### BRIEF DESCRIPTION OF THE DRAWINGS

The accompanying drawings illustrate implementations of the devices and methods disclosed herein and together with the description, serve to explain the principles of the present disclosure.

FIG. 1 illustrates an exemplary system for staging a pressure injury according to implementations of the present disclosure.

FIG. 2A illustrates a flow chart for staging a pressure injury according to implementations of the present disclosure.

FIG. 2B illustrates a flow chart for staging a pressure injury according to implementations of the present disclosure.

FIG. 2C illustrates a flow chart for staging a pressure injury according to implementations of the present disclosure.

FIG. 3 illustrates an exemplary system for staging a pressure injury according to implementations of the present disclosure.

FIG. 4 illustrates an exemplary screen of a system for staging a pressure injury according to implementations of the present disclosure.

The accompanying drawings may be better understood by reference to the following detailed description.

### DETAILED DESCRIPTION

For the purposes of promoting an understanding of the principles of the present disclosure, reference will now be made to the implementations illustrated in the drawings. Specific language will be used to describe the same. It will nevertheless be understood that no limitation of the scope of the disclosure is intended. Any alterations and further modifications to the described devices, systems, methods, and any further application of the principles of the present disclosure are fully contemplated as would normally occur to one skilled in the art to which the disclosure relates. In particular, it is fully contemplated that the features, components, and/or steps described with respect to one implementation may be combined with the features, components, and/or steps described with respect to other implementations of the present disclosure. For simplicity, in some instances the same reference numbers are used throughout the drawings to refer to the same or like parts.

The present disclosure relates generally to systems, devices, and methods relating to assessing and staging pressure injuries. The present disclosure may offer benefits over existing methods for staging pressure injuries.

First, the implementations presented here may assist a clinician in correctly staging the pressure injury by utilizing the criteria/definitions listed in the National Pressure Injury Advisory Panel's definitions of the staging of pressure injuries while eliminating time wasted by reviewing irrelevant information. In particular, these implementations serve to direct a clinician to a correct assessment of a pressure injury using intuitive yes or no answers to simple questions.

Second, the implementations provided here serve to provide a reviewable history a staging procedure. That is, the questions provided to a clinician and answers received are displayed in a visual format to ensure that they have been answered correctly. The identifications and treatment options referred to herein may be similar to those provided by the National Pressure Ulcer Advisory Panel, European Pressure Ulcer Advisory Panel and Pan Pacific Pressure Injury Alliance, “Prevention and Treatment of Pressure Ulcers: Quick Reference Guide” (Emily Haesler (Ed.), Cambridge Media: Osborne Park, Western Australia; 2014) and Rodriguez, R., Fullana-Matas, A., Miro′-Bonet, R., (2021), “Classification of Pressure Injury Stages and Skin Damage Photographs by RNs and Nursing Students in Spain: An Observational Comparison Study.” (Journal of Wound, Ostomy and Continence Nursing, September/October 2021, Volume 48, Number 5), both of which are incorporated in their entirety by reference.

Therefore, the present disclosure meets the existing needs for an intuitive, efficiently pressure staging method that may be used by new or seasoned clinicians alike.

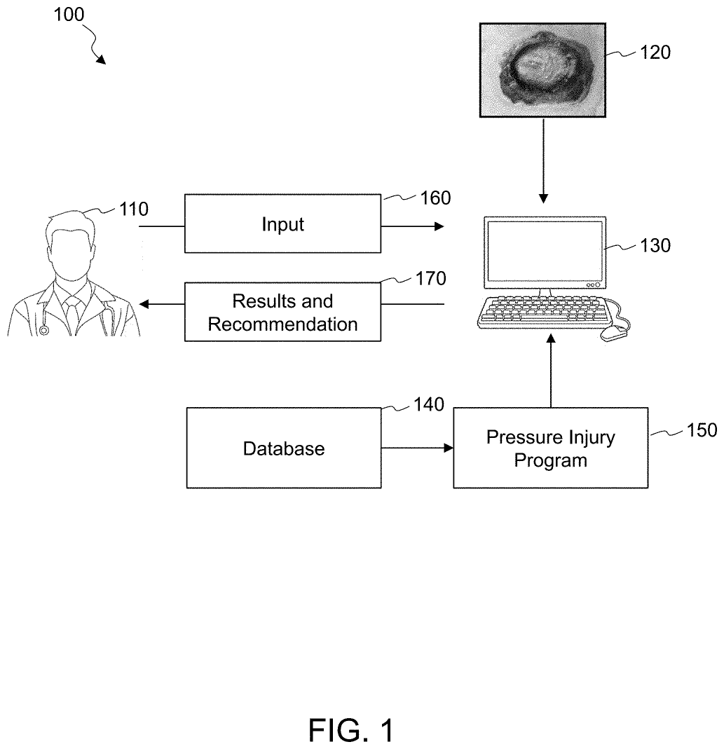

FIG. 1 illustrates an illustration of an exemplary system 100 for staging pressure injuries. A clinician 110 (who may include healthcare professionals, physicians, physician assistants, nurse practitioners, nurses, and therapists) may need to assess a pressure injury. A visual depiction 120 of the injury may be displayed to the clinician 110 on a screen such as on computer 130 or other medical device. In some implementations, the visual depiction includes a scale or other size reference as well as coloration to aid in its identification. The clinician may also be provided with a history of the injury and accompanying information about the patient with the injury.

The clinician 110 upon observing the visual depiction 120 may access the pressure injury application 150. As discussed below, the pressure injury application 150 may include an algorithm or flow chart to assist the clinician in assessing the visual depiction 120 of the injury. The pressure injury application 150 may include a number of easily understandable questions that guide the clinician 110 to an identification of the injury based on yes or no answers. This application 150 may include various visual aspects, such as color coded decisions based on previous answers, an overall flow chart showing all of the previous answers given, and a result image.

To conduct a staging process, a clinician 110 may access the pressure injury application 150 such by a computer 130 or other medical device. The pressure injury application 150 provides various prompts which the clinician 110 answers with simple answers, such as through an input device such as a mouse, keyboard, touchscreen, or other input device. Based on these answers, the pressure injury application 150 gives further prompts until the injury has been identified. This injury identification is then provided to the clinician 110. In some implementations, the injury identification is accompanied by a prompt to compare the identification to other medical data, such as a NPIAP definition of the pressure injury stage associated with the identification. The pressure injury application 150 may optionally give a recommendation based on the injury identification. This recommendation may include further steps for identifying the injury as well as treatment options. The clinician 110 may then take action to assess or treat the pressure injury based on these recommendations.

The pressure injury application 150 may access a database 140 with various medical data, including detailed written descriptions and visual depictions (including photographs) of tissue types, detailed written descriptions and visual depictions (including photographs) of wound types, and NPIAP definitions of the pressure injury stages. These medical data may be provided by the pressure injury application 150 in response to answers from the clinician 110, as well as upon completion of the injury assessment. In some implementations, the pressure injury application 150 may automatically display medical data during the assessment, such as NPIAP definitions along with a prompt for the clinician 110 as a check.

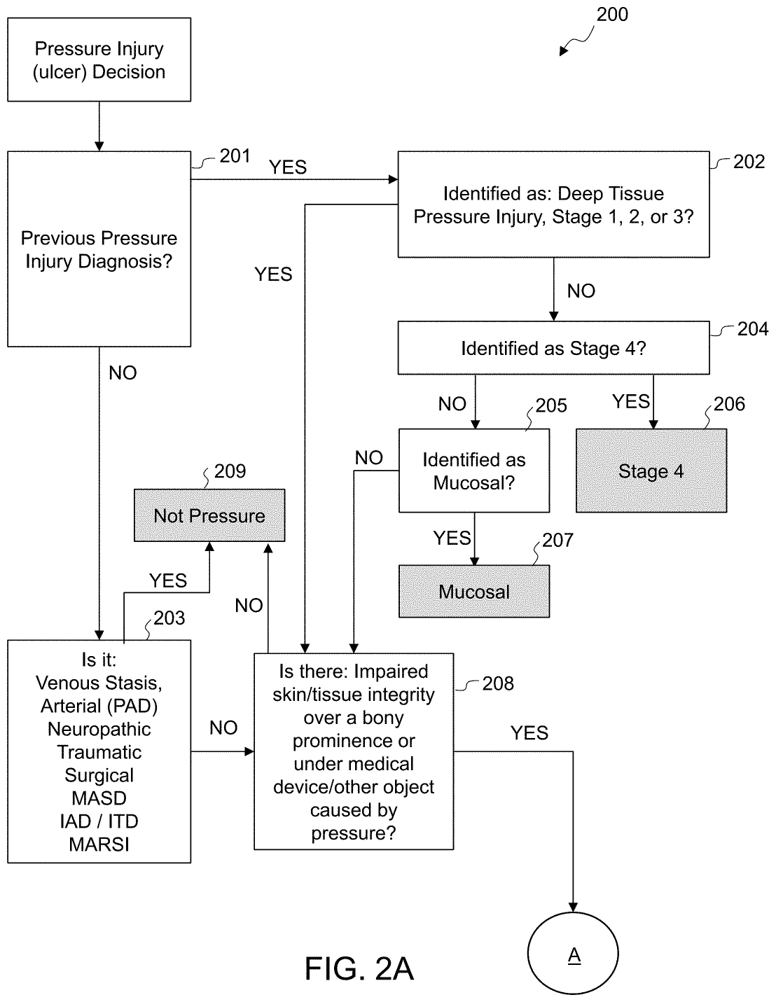

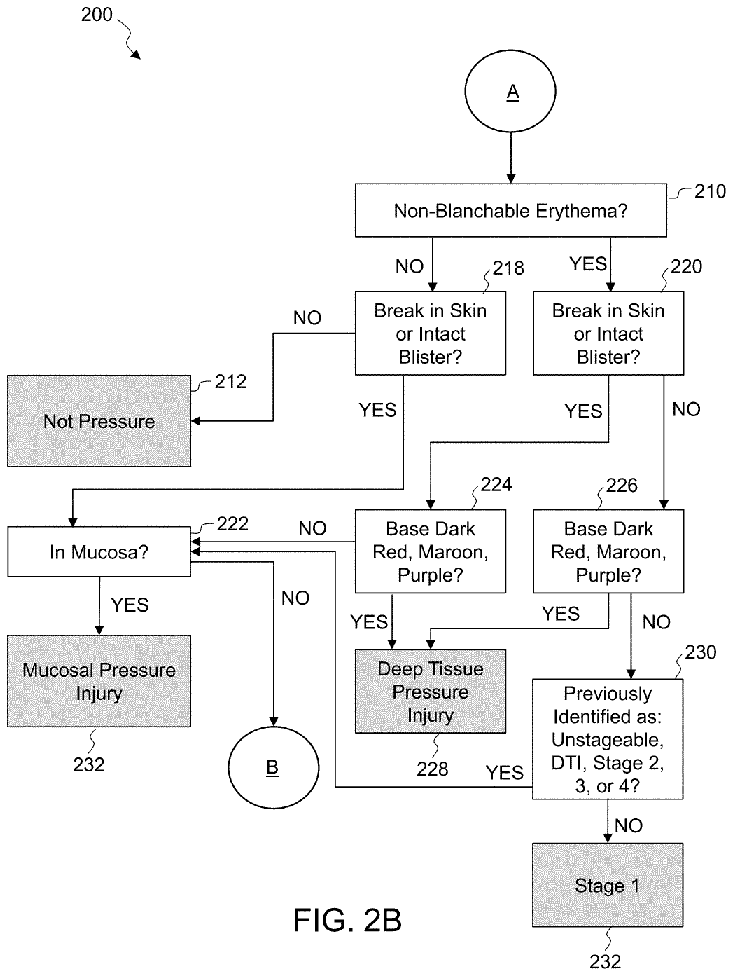

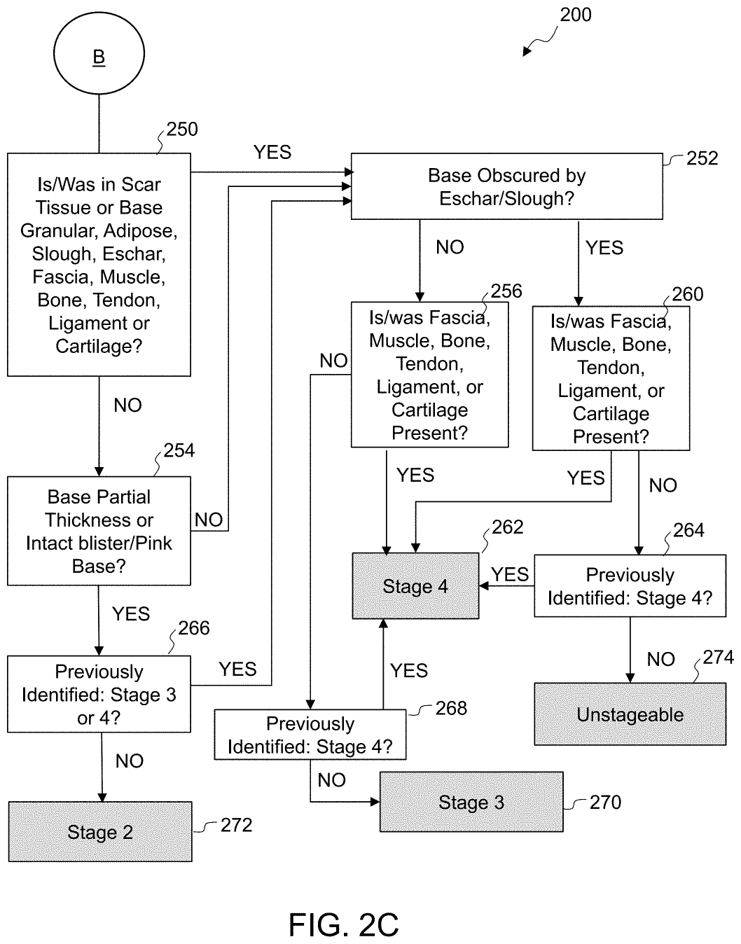

FIG. 2A illustrates a flow chart 200 for staging a pressure injury according to implementations of the present disclosure. In some implementations, the flow chart is part of the pressure injury application 150 shown in FIG. 1. The flow chart 200 may be configured to guide a clinician 110 through a number of simple questions to quickly and accurately assess a pressure injury. The flow chart 200 may include a number of questions or prompts (which a clinician 110 may be prompted to answer with a yes or no answer) and assessments (which may provide an identification of the pressure injury). For ease of understanding, the assessments have boxes that are shaded compared to the questions or prompts which have boxes that are unshaded.

The flow chart 200 may begin with a Pressure Injury (ulcer) Decision. The first question 201 is if there was a previous pressure injury diagnosis. The clinician may select yes or no to this question 210 to proceed through the flow chart 200. If the answer to question 201 is yes, the flow chart 200 proceeds to question 202 of if the injury was identified as a deep tissue pressure injury, stage 1, 2, or 3? If the answer to question 201 is no, the flow chart 200 may proceed to question 203 of if the injury is one of Venous Stasis, Arterial (PAD), Neuropathic, Traumatic, Surgical, Moisture Associated Skin Damage (MASD), Incontinence Associated Dermatitis (IAD) or Intertriginous Dermatitis (ITD), or Medical Adhesive Related Skin Injury (MARSI)? If the answer to question 203 is yes, the flow chart 200 proceeds to assessment 209 of not a pressure injury. If the answer to question 202 is yes or the answer to question 203 is no, the flow 200 may proceed to question 208 of is there impaired skin/tissue integrity over a bony prominence or under medical device/other object caused by pressure? If the answer to question 208 is no, the flow chart 200 may proceed to an assessment 209 of not pressure. If the answer to question 202 is no, the flow chart 200 may proceed to question 204 of if the injury was identified as stage 4? If the answer to question 204 is yes, the flow chart 200 may proceed to an assessment of stage 4. If the answer to question 204 is no, the flow chart 200 may proceed to question 205 of if the injury was identified as mucosal? If the answer to question 205 is yes, the flow chart 200 may proceed to assessment 207 of mucosal. If the answer to question 205 is no, the flow chart 200 may proceed to question 208. If the answer to question 208 is yes, the flow chart 200 may continue to reference numeral A to continue the flow chart in FIG. 2B.

FIG. 2B continues with a question 210 of is the pressure injury Non-Blanchable Erythema? If the answer to question 210 is no, the flow chart 200 proceeds to question 218 which is there is a break in skin or intact blister? If the answer to question 210 is yes, the flow chart 200 proceeds to question 220 which is there is a break in skin or intact blister? If the answer to question 218 is no, the flow chart 200 proceeds to assessment 212 of “not pressure.”

If the answer to question 218 is yes, the flow chart 200 proceeds to question 222 which is the injury in mucosa? If the answer to question 220 is yes, the flow chart 200 proceeds to question 224 which is the base dark red, maroon or purple? If the answer to question 220 is no, the flow chart 200 proceeds to question 226 which is the base dark red, maroon or purple? If the answer to question 224 is no, the flow chart 200 proceeds to question 222 which is the injury in mucosa? If the answer to question 224 is yes, the flow chart 200 proceeds to assessment 228 of a deep tissue pressure injury. If the answer to question 226 is yes, the flow chart 200 proceeds to assessment 228 of a deep tissue pressure injury. If the answer to question 226 is yes, the flow chart 200 proceeds to assessment 228 of a deep tissue pressure injury. If the answer to question 226 is no, the flow chart 200 proceeds to question 230 of if the injury was previous identified as unstageable, DTI, Stage 2, 3, or 4? If the answer to question 230 is no, the flow chart 200 may proceed to assessment 232 of a stage 1 pressure injury. If the answer to question 222 is yes, the flow chart 200 proceeds to assessment 232 of a mucosal pressure injury. If the answer to question 230 is yes, the flow chart 200 may proceed to question 222. If the answer to question 222 is no, the flow chart 200 proceeds to reference numeral B which continues the flow chart in FIG. 2C.

FIG. 2C illustrates a continuation of flow chart 200 for staging a pressure injury according to implementations of the present disclosure. In particular, reference numeral B continues to question 250 which is/was in the pressure injury in scar tissue or base granular, adipose, slough, eschar, fascia, muscle, bone, tendon, ligament or cartilage? If the answer to question 250 is no, the flow chart 200 proceeds to question 254 which is the base partial thickness or intact blister or pink base? If the answer to question 250 is yes, the flow chart 200 proceeds to question 252 which is the base obscured by eschar or slough? If the answer to question 254 is yes, the flow chart 200 proceeds to question 266 of if the injury was previously identified as stage 3 or 4? If the answer to question 266 is no, the flow chart 200 may proceed to assessment 272 stage 2 pressure injury. If the answer to question 266 is yes, the flow chart may proceed to question 256.

If the answer to question 254 is no, the flow chart 200 proceeds to question 256 which is there fascia, muscle, bone, tendon, ligament, or cartilage present? If the answer to question 252 is yes, the flow chart 200 proceeds to question 260 which is there fascia, muscle, bone, tendon, ligament, or cartilage present? If the answer to question 256 is no, the flow chart 200 proceeds to assessment 268 which is a stage 3 pressure injury. If the answer to question 256 is yes, the flow chart 200 proceeds to assessment 262 which is a stage 4 pressure injury. If the answer to question 260 is yes, the flow chart 200 proceeds to question 264 of if the injury was previously identified as stage 4? If the answer to question 264 is yes, the flow chart 200 may proceed to assessment 262 which is a stage 4 pressure injury. If the answer to question 260 is no, the flow chart 200 proceeds to assessment 274 which is that the pressure injury is unstageable.

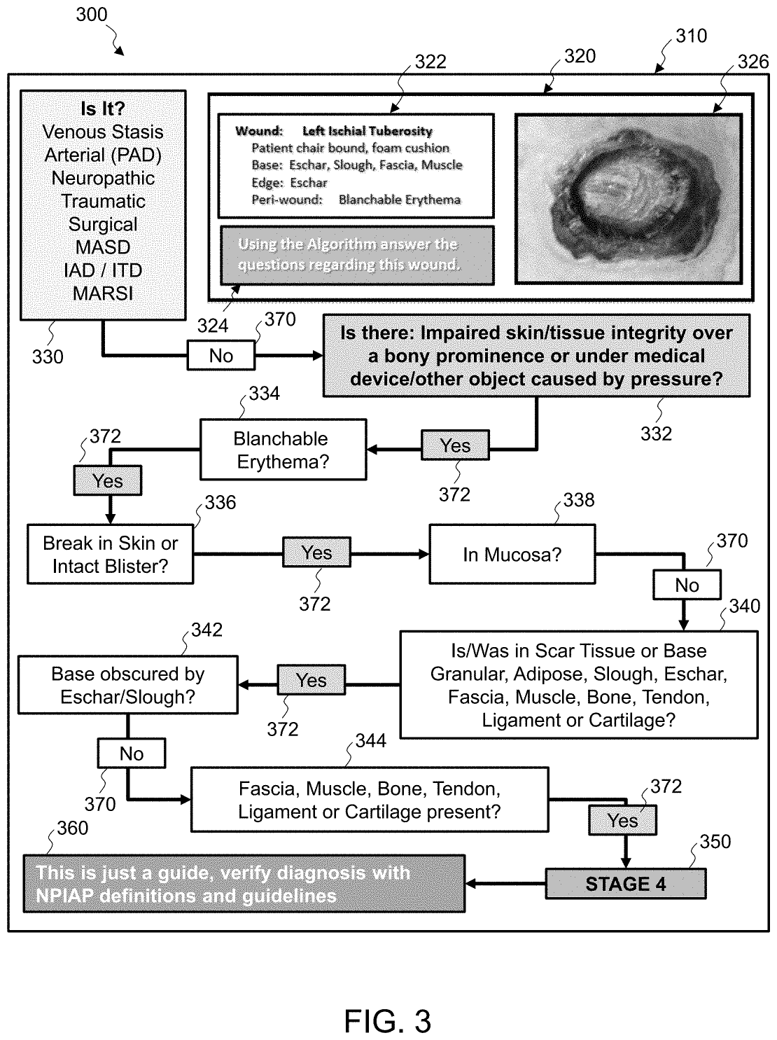

FIG. 3 illustrates an exemplary system 300 for staging a pressure injury according to implementations of the present disclosure. In some implementations, the system 300 includes a display 310 which shows a visual depiction of a pressure injury application, such as pressure injury application 150 show in FIG. 1. In some implementations, the display 310 is displayed to a clinician 110 on a computer screen or other medical device screen. The system 300 may include an injury viewing area which may include a visual depiction 326 of a pressure injury along with accompanying medical information 322 and a prompt 324 to identify the pressure injury using the pressure injury application or algorithm.

To conduct a staging procedure, the clinician 110 may be provided with a history of the pressure injury and may examine the wound prior to consulting the pressure injury application. The clinician is then prompted to answer a series of questions. These questions may be the questions of the flowchart 200 of FIGS. 2A and 2B and may proceed according to the answers given in the flowchart 200. For example, the clinician may be prompted to begin with an initial question 330. In some implementations, this question 330 is whether this injury is Venous Stasis, Arterial (PAD), Neuropathic, Traumatic, Surgical, MASD, IAD/ITD, or MARSI. The initial question 330 may be displayed with a particular color such as green to make it clear to the clinician 110 where to start. The clinician answers this question (in this case no) and the procedure continues to the next question and so on until an assessment of the pressure injury is given as shown in FIG. 3.

In the example of FIG. 3, all aspects of the procedure are visually displayed, such that all of the question boxes 332, 334, 336, 338, 340, 342, 344, answer icons 370, 372, and assessment box 350 are displayed on the single screen 310. The various questions and answers may be connected by arrows to intuitive show the path through the flow chart. This may help a clinician to visualize the procedure more quickly and track his or her route through the flow chart. This complete view may also be accessed by a supervisor or other clinician to check each step of the staging process and quickly check the answers given to ensure accuracy of the result. The various question boxes 332, 334, 336, 338, 340, 342, 344, answer icons 370, 372, and assessment box 350 may be color coded for ease in understanding the procedure, such as having different colors for question boxes, 332, 334, 336, 338, 340, 342, 344, answer icons 370, 372 (including different colors for yes and no as shown) and assessment box 350.

The clinician may be able to quickly look back through the history of the procedure to check the various answers given. It may also allow a supervisor or other clinician to quickly assess the procedure for accuracy. In some implementations, the various steps taken through the flowchart are visually shown to track the progress of the clinician 110. This may help the clinician to quickly view the possible options given and more quickly and accurately assess the injury.

In some implementations, only the presented questions and given answers are shown on the screen 310. In other implementations, other questions and answers are shown, such as the entire flowchart along with “chosen” route through the flowchart. For example, the screen 310 may display questions other than those presented to the clinician as well as possible answers and corresponding flows corresponding to those paths. The “chosen” path relevant to assessing the pressure injury may be highlight in some way to distinguish it from other options, such as having bold text or different colors than the other options. This may help the clinician to visualize other alternative paths through the flowchart and may be used for education purposes.

In some implementations, the assessment box 350 that is provided based on all of the given questions and received answers from the clinician 110 is accompanied by a prompt 360 that prompts the clinician to compare the assessment provided with selected medical data, such as a NPIAP definition corresponding to the assessment. This may help the clinician 110 to quickly visually compare the assessment result with the provided pressure injury visualization. The clinician is able to “verify” the wound type with the NPIAP definitions. The clinician or their designee may then apply the treatment appropriate for the identified pressure injury stage according to the standard of care.

Other visualizations may be automatically provided during the procedure, such as being provided to accompany the various questions asked. In other implementations, these visualizations are provided as a selectable button along with the question such that the clinician 110 can quickly access the visualization if he or she is unsure of the correct answer. These visualization may provide example medical images, definitions, or links to other information sources.

In some implementations, other information is gathered by the system 300 and displayed on the screen 310, such as a timestamp when the answers were answered and/or the name or ID number of a specific clinician 110 answering the questions. The system 300 may also allow a clinician 110 to quickly access and change previous questions, such as providing a back button as well as making the questions selectable after answers are given. In this way, a clinician 110 can quickly backtrack and receive a different assessment result if an error is found.

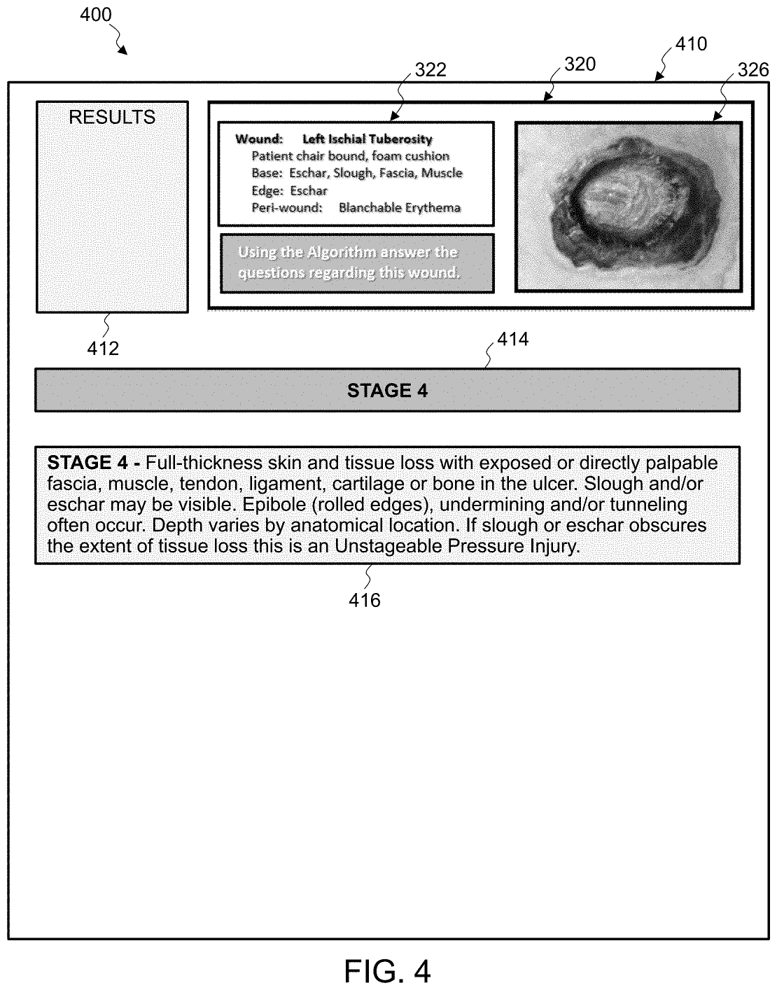

FIG. 4 is an exemplary display 400 that may be shown on the system 300 with an end result of the assessment. In some implementations, this end result display 400 may include the assessment given in the assessment box 350 of FIG. 3 along with a medical definition 416 for reference. The use of system 300 and the end result display with the full medical definition 416 may allow a clinician to avoid having to read irrelevant medical definitions and more quickly come to an accurate result, while still providing a final check of the pressure injury assessment. The medical definition 416 may be accompanied by a visualization, such photographs showing similar pressure injuries.

Persons of ordinary skill in the art will appreciate that the implementations encompassed by the present disclosure are not limited to the particular exemplary implementations described above. In that regard, although illustrative implementations have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. A method for staging a pressure injury, comprising:
providing a visualization of the pressure injury to a clinician;
providing a series of questions to the clinician;
receiving answers to each of the series of questions from the clinician;
providing an assessment of the pressure injury to the clinician based on the provided series of questions and corresponding received answers from the clinician;
displaying the provided series of questions and corresponding received answers along with the assessment of the pressure injury in a visual format on a single screen of a display device.

2. The method of claim 1, wherein the visual format is a flowchart such that each of the provided series of questions is displayed in a first set of boxes and each of the corresponding received answers is displayed in a second set of boxes.

3. The method of claim 2, wherein the first set of boxes and the second of boxes are connected by arrows to visually show a path of the questions and answers through the flowchart.

4. The method of claim 1, wherein the assessment of the pressure injury is provided visually along with a prompt for the clinician to access a medical definition corresponding to the assessment of the pressure injury.

5. The method of claim 4, wherein the assessment of the pressure injury is provided with a medical visualization corresponding to the assessment of the pressure injury.

6. The method of claim 1, wherein the provided series of questions is displayed as a set of selectable icons.

7. The method of claim 1, wherein the received answers to the questions are displayed as a set of selectable icons.

8. The method of claim 1, wherein the provided series of questions is in a yes or no format.

9. The method of claim 1, further comprising providing a medical recommendation based on the assessment of the pressure injury.

10. The method of claim 9, further comprising displaying the provided medical recommendation based on the assessment of the pressure injury with the assessment of the pressure injury in a visual format on the single screen of the display device.

11. A system for staging a pressure injury, comprising:
an electronic computer system configured to receive medical data and user input information, wherein the electronic computer system is configured to receive a visualization of the pressure injury, wherein the electronic computer system is configured to generate a series of questions related to assessing the visualization of the pressure injury;
an input device for a user to input the information connected to the electronic computer system, wherein the input device is configured to receive answers to series of questions related to the assessing of the visualization of the pressure injury; and
a display device configured to display the medical data and user input information in a visual format; wherein the display device is configured to display the series of questions and the received answers in a visual format on a single screen of the display device.

12. The system of claim 11, wherein the visual format is a flowchart such that each of the provided series of questions is displayed in a first set of boxes and each of the corresponding received answers is displayed in a second set of boxes.

13. The system of claim 12, wherein the first set of boxes and the second of boxes are connected by arrows to visually show a path of the questions and answers through the flowchart.

14. The system of claim 11, wherein the assessment of the pressure injury is provided visually along with a prompt for the clinician to access a medical definition corresponding to the assessment of the pressure injury.

15. The system of claim 14, wherein the assessment of the pressure injury is provided with a medical visualization corresponding to the assessment of the pressure injury.

16. The system of claim 11, wherein the provided series of questions is displayed as a set of selectable icons.

17. The system of claim 11, wherein the received answers to the questions are displayed as a set of selectable icons.

18. The system of claim 11, wherein the provided series of questions is in a yes or no format.

19. A method for identifying a pressure injury, comprising:
providing a visualization of the pressure injury to a clinician;
providing a first question in a first icon to the clinician;
receiving a first answer to the first question from the clinician;
displaying the first answer to the first question in a second icon;
providing a second question in a third icon to the clinician based on the first answer to the first question;
receiving a second answer to the second question from the clinician;
displaying the second answer to the second question in a fourth icon;
providing an assessment of the pressure injury based on the provided first and second questions and received first and second answers; and
displaying the assessment of the pressure injury in a visual format along with the first, second, third, and fourth icons on a single screen of a display device.

20. The method of claim 19, further comprising displaying the assessment of the pressure injury in the visual format with the first, second, third, and fourth icons in a flowchart such that adjacent icons are connected visually to show an order of questions and answers.
